(19) United States
(12) Patent Application Publication
(10) Pub. No.: US 2023/0122646 A1
(43) Pub. Date: Apr. 20, 2023

(54) ANTIGENIC DETECTOR

(71) Applicant: GUANGDONG BIOALL MEDICAL TECHNOLOGY CO., LTD., Dongguan (CN)

(72) Inventor: Yuean HU, Dongguan (CN)

(21) Appl. No.: 17/720,004

(22) Filed: Apr. 13, 2022

(30) Foreign Application Priority Data
Oct. 19, 2021 (CN) 202122509654.5

(51) Int. Cl. G01N 33/569; B01L 3/00; G01N 33/543

(52) U.S. Cl. CPC G01N 33/56983; B01L 3/5023; G01N 33/54388; B01L 2300/0832; B01L 2300/069; B01L 2200/16; B01L 2400/0683; B01L 2300/0672; G01N 2333/165

(57) ABSTRACT

The present application discloses an antigenic detector, including: a pen holder and a pen cap. The pen holder includes: a test paper tube, a test paper assembled in the test paper tube, a test paper support, and a foam support installed on the front end of the test paper tube with a foam and a protective cover; the pen cap includes: a base, an aluminum foil assembled in the base, and an interior of the base forms a cavity into which a front end of the pen holder is inserted, and a reaction solution is sealed inside the cavity of the base by the aluminum foil; the protective cover is detachably installed on the front end of the test paper tube and covers the foam; the foam support is provided with a channel that communicates with an interior of the test paper tube.

### CROSS-REFERENCE TO RELATED APPLICATIONS

This application claims priority to Chinese Patent Application No. 202122509654.5 filed on Oct. 19, 2021, the contents of which are incorporated herein by reference in their entirety.

### BACKGROUND

### Technical Field

The present application relates to the technical field of an antigenic detector.

### Description of Related Arts

At present, the detection of novel coronavirus infection focuses on the detection of novel coronavirus IgG and IgM antibodies in the blood of suspected patients, but the detection products directly targeting novel coronavirus antigens are still rare; and most testing products of the detection of other viral antigens are aimed at blood testing. This blood testing method is not suitable for home use and cannot be tested by itself.

Therefore, the applicant has developed an antigenic detector with a simple structure and convenient use.

### SUMMARY

In view of this, the present application provides an antigenic detector for detection based on saliva.

In order to achieve the above purpose, the present application adopts the following technical scheme: an antigenic detector, including: a pen holder and a pen cap. The pen holder includes a test paper tube, a test paper assembled in the test paper tube, a test paper support, and a foam support installed on the front end of the test paper tube with a foam and a protective cover. The pen cap includes a base, an aluminum foil assembled in the base, and an interior of the base forms a cavity into which a front end of the pen holder is inserted, and a reaction solution is sealed inside the cavity of the base by the aluminum foil. The protective cover is detachably installed on the front end of the test paper tube and covers the foam. The foam support is provided with a channel that communicates with an interior of the test paper tube. A liquid absorbed by the foam is mixed with the reaction liquid and is transferred to the test paper inside the test paper tube through the channel.

In a further technical solution, the test paper tube is in the shape of a hollow tube, and the test paper support is arranged inside the test paper tube, and the test paper is installed on test paper support. The end of the test paper tube is provided with a test tube cover, and an air hole communicating with the interior of the test paper tube is defined on the test tube cover.

In a further technical solution, the foam support includes: an insertion end inserted into the test paper tube and a mounting end extending out of the test paper tube, the foam is mounted on the mounting end, and the foam at least partially protrudes out of the test paper tube.

In a further technical solution, an end of the mounting end is a pointed structure.

In a further technical solution, a rubber stopper is arranged inside the test paper tube, and the rubber stopper holds the test paper separated from foam support when not in use.

In a further technical solution, the base includes: an upper cover and a lower cover, and a boss is formed on the lower cover, and the boss is provided with a sealing ring, and the lower cover is inserted into the upper cover through the boss.

In a further technical solution, the foam support is provided with an O-ring, and the O-ring is in contact with an inner wall of the cavity of the base.

In a further technical solution, the aluminum foil is supported on the boss. When the upper cover and the lower cover are combined, the aluminum foil is stably clamped, or the aluminum foil is hot-pressed on the boss, or the aluminum foil is ultrasonically fixed on the boss; or the aluminum foil is glued to the boss.

In a further technical solution, the test paper tube is provided with an observation window or the test paper tube is arranged to be transparent.

In a further technical solution, the insertion end of the foam support is provided with an undercut, and a buckle is arranged inside the test paper tube corresponding to the undercut to prevent the foam support from being pulled out of the test paper tube.

As can be seen from the above-mentioned technical solutions, compared with the related art, the present application has the following advantages:

Before use, the antigenic detector of the present application can form a relatively sealed space for the test paper to protect the test paper, and at the same time, the front end of the pen holder is provided with a protective cover to protect the foam; the foam absorbs saliva quickly; the pen holder is inserted into the pen cap to pierce the aluminum foil at the front of the pen holder, the foam absorb the reaction solution, the reaction solution is mixed with the saliva, the mixture is squeezed into the pen holder, react with the test paper, and the response of the test paper is observed through the test paper tube and the results obtained are simple and convenient to use.

A sealing ring is arranged between the pen holder and the pen cap of the present application, which will not be exposed when the saliva is squeezed. At the same time, after the use is completed, since the pen holder and the pen cap are relatively airtight, which will not cause pollution after being discarded. Saliva will not evaporate in the air, the used antibiotic pen can be collected and processed simply.

### BRIEF DESCRIPTION OF THE DRAWINGS

In order to more clearly illustrate the embodiments of the present application or the technical solutions in the prior art, the following briefly introduces the accompanying drawings that need to be used in the description of the embodiments or the prior art. Obviously, the accompanying drawings in the following description are only an embodiment of the present application, and for those of ordinary skill in the art, other drawings can also be obtained according to the provided drawings without creative efforts.

FIG. 1 is a schematic diagram of the present application;

FIG. 2 is a schematic diagram of the pen holder in the present application;

FIG. 3 is an exploded view of the pen holder in the present application;

FIG. 4 is an exploded view of pen cap in the present application;

FIG. 5 is a cross-sectional view of the pen holder not completely inserted into the pen cap during use; and

FIG. 6 is a cross-sectional view of the pen holder completely inserted into the pen cap during use.

### DETAILED DESCRIPTION

The present application will now be described in further detail with reference to the accompanying drawings. These drawings are all simplified schematic diagrams, and only illustrate the basic structure of the present application in a schematic manner, so they only show the structures related to the present application.

In the description of this application, it should be understood that the orientation or positional relationship indicated by the terms of “longitudinal”, “radial”, “length”, “width”, “thickness”, “upper”, “lower”, “left”, “right”, ““front”, “rear”, “vertical”, “horizontal”, “top”, “bottom”, “inside”, “outside”, etc. is based on the orientation or positional relationship shown in the accompanying drawings. It is only for the convenience of describing the present application and simplifying the description, rather than indicating or implying that the referred device or element must have a particular orientation, be constructed and operate in a particular orientation, and therefore should not be construed as a limitation of the present application. In the description of this application, unless stated otherwise, “a plurality of” means two or more.

In the description of this application, it should be noted that, unless otherwise expressly specified and limited, the terms “installed”, “connected” and “connecting” should be understood in a broad sense, for example, it may be a fixed connection or a detachable connection, or integral connection; it can be mechanical connection or electrical connection; it can be directly connected or indirectly connected through an intermediate medium, and it can be internal communication between two elements. For those of ordinary skill in the art, the specific meanings of the above terms in this application can be understood in specific situations.

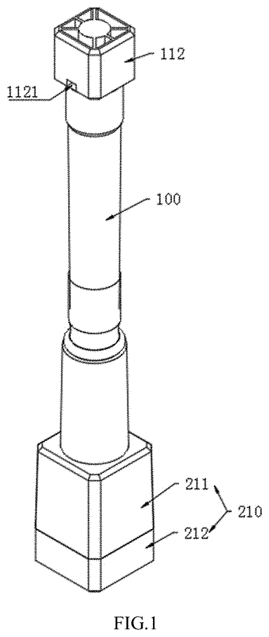

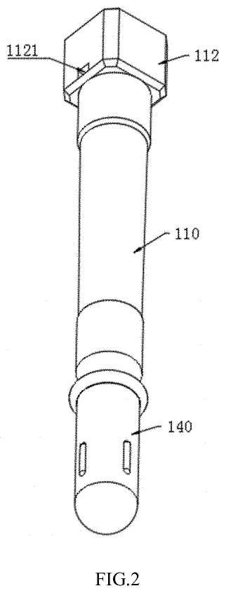

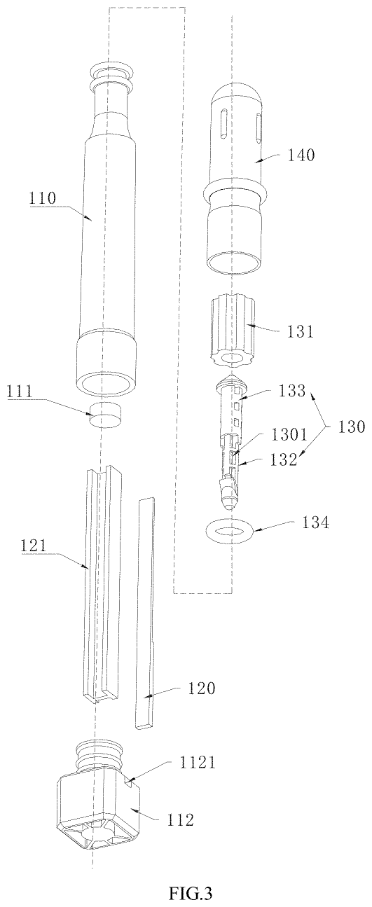

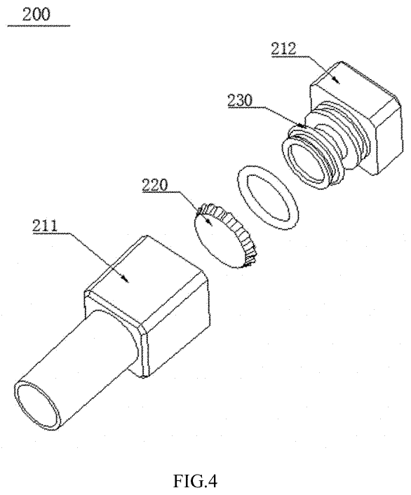

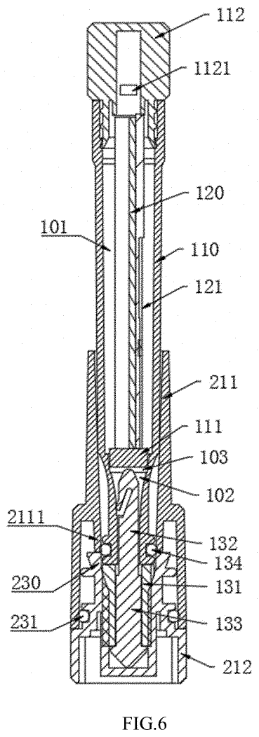

Referring to FIGS. 1-6, the antigenic detector includes: a pen holder 100 and a pen cap 200, the pen holder 100 is matched with the pen cap 200.

The pen holder 100 includes: a test paper tube 110, a test paper 120, a foam support 130, a foam 131, and a protective cover 140.

The test paper tube 110 is roughly in the shape of a tube, the test paper tube 110 is hollow, and a test tube cover 112 is installed at the end of the test paper tube 110. The test paper 120 is stably installed in the test paper tube 110 through test paper supports 121. During installation, the test tube cover 112 is disassembled, after the test paper 120 is placed on the test paper supports 121, the test paper supports 121 are put into the test paper tube 110, and then the test tube cover 112 is installed on the end of the test paper tube 110; the test paper supports 121 are provided by a placement groove where the test paper 120 is placed, and the test tube cover 112 is provided with an air hole 1121. The air hole 1121 is configured for the purpose of discharging the air in the test paper tube 110 when the subsequent mixed liquid (saliva+reaction solution) is squeezed into the test paper tube 110, the air inside the test paper tube 110 is discharged and the air pressure is reduced so that the mixing liquid can enter the test paper tube 110 smoothly.

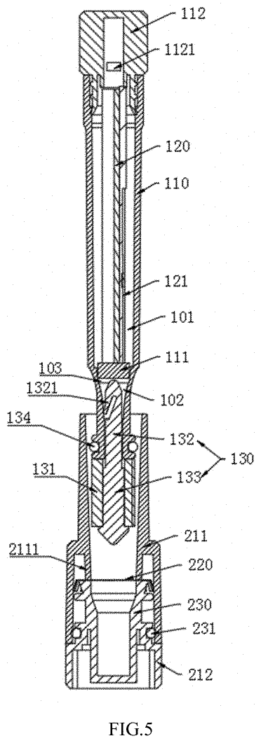

The foam support 130 includes: an insertion end 132 inserted into the test paper tube 110 and a mounting end 133 extending out of the test paper tube 110, the foam 131 is mounted on the mounting end 133, and the foam 131 at least partially extends out of the test paper tube 110. As shown in FIG. 5, after installation, the foam 131 is located outside the test paper tube 110 and abuts with the front end of the test paper tube 110; the end of the mounting end 133 is a pointed structure, and the cross-sectional area of the pointed structure is larger than that of the mounting end 133, so that when the foam 131 is installed, the foam 131 does not be separated from the front end of the foam support 130. The pointed structure is also used to pierce the aluminum foil 220 later.

The foam support 130 is provided with a channel that communicates with the interior of the test paper tube 110, and the liquid absorbed by the foam 131 is transferred to the test paper 120 in the test paper tube 110 through the channel. The outer wall of the foam support 130 is provided with a plurality of openings 1301 communicating with the channels.

The diameter of the front end of the test paper tube 110 is smaller than the diameter of the rear end of the test paper tube 110, which is convenient for inserting into the base 210.

The protective cover 140 is detachably installed on the front end of the test paper tube 110 and covers the foam 131. Before use, the protective cover 140 is installed on the front end of the test paper tube 110 to protect the foam 131. When needed, the protective cover 140 can be removed for use.

Since the diameter of the front end of the test paper tube 110 is smaller than the diameter of the rear end, the interior of the test paper tube 110 is divided into a first cavity 101 and a second cavity 102. The first cavity 101 is used to install the test paper supports 121, and the second cavity 102 is used to install the foam support 130. A rubber stopper 111 is installed between the first cavity 101 and the second cavity 102. When not in use, the rubber stopper 111 separates the test paper 120 from the foam support 130.

The arrangement of the rubber stopper 111 is not fixed. The junction of the first cavity 101 and the second cavity 102 constitutes a mounting position 103 of the rubber stopper 111. When not in use, the rubber stopper 111 is installed in the mounting position, and the first cavity 101 and the second cavity 102 is separated; the side wall of the mounting position 103 is arc-shaped, so that after the rubber stopper 111 is pushed back by the foam support 130, the first cavity 101 and the second cavity 102 are connected.

The insertion end 132 of the foam support 130 is provided with an undercut 1321, and the test paper tube 110 is provided with a corresponding buckle. When the foam support 130 is inserted into the test paper tube 110 smoothly, the undercut 1321 of the foam support 130 cooperates with the buckle to prevent the foam support 130 from being pulled out of the test paper tube 110.

Before the liquid enters the test paper tube 110, the foam support 130 is not completely inserted into the test paper tube 110, and a certain insertion stroke is reserved. When testing is required, the front end of the foam support 130 contacts with the inner bottom wall of the pen cap 200 to push the foam support 130 move inward, and finally the rubber stopper 111 is pushed inward, so that the mixture can enter the first cavity 101.

The test paper tube 110 is provided with an observation window or the test paper tube 110 is transparently arranged, so as to facilitate the observation of changes of the test paper 120.

The pen cap 200 includes: a base 210, an aluminum foil 220 assembled in the base 210, and the interior of the base 210 constitutes a cavity for the front end of the pen holder 100 to be inserted.

The base 210 includes: an upper cover 211 and a lower cover 212, a boss 230 is formed on the lower cover 212, a sealing ring 231 is provided on the boss 230, and the sealing ring 231 is sealed between the upper cover 211 and the lower cover 212; the lower cover 212 is inserted into the upper cover 211 through the boss 230, and the aluminum foil 220 of the lower cover 220 is supported on the boss 230. The aluminum foil 220 is a thin film structure, and the reaction solution is sealed inside the cavity of the base 210 through the aluminum foil 220. The aluminum foil 220 is installed on the top of the boss 230, and the reaction solution is sealed. When the pen holder 100 is inserted into the pen cap 200, the foam support 130 at the front end of the pen holder 100 pierces the aluminum foil 220, then the foam 131 absorbs the reaction solution in the base 210, and the reaction solution is mixed with saliva.

Furthermore, a ring of raised ribs 2111 is formed on the inner wall of the upper cover 211. The raised ribs 2111 cooperate with the boss 230 on the lower cover 212. When the lower cover 212 is installed on the upper cover 211, the boss 230 and the raised ribs 2111 are pressed up and down, and the aluminum foil 220 is sandwiched between the boss 230 and the raised ribs 2111;

In other technical methods, the aluminum foil 220 can also be fixed on the boss 230 by hot pressing, or fixed on the boss 230 by ultrasonic fixing, or installed on the boss 230 with glue; the aluminum foil 220 can also be fixed on the boss 230 in any other easy and alternative way.

The foam support 130 is provided with an O-ring 134, and the O-ring 134 contacts with the inner wall of the cavity of the base 210.

The diameter of the cavity in the base 210 is reduced from the opening to the inner diameter, which is convenient for squeezing the foam 131 through the inner wall of the cavity, and the O-ring 134 on the foam support 130 is matched with the cavity wall to prevent the liquid from overflowing the base 210 in the process of squeezing, which ensures the safety and hygiene of use, and also prevents infection.

The use of this product is further explained below:

Before use, the pen cap 200 is separated from the pen holder 100, and the protective cover 140 is installed on the front end of the test paper tube 110;

When it needs to be used, the protective cover 140 is removed at the front end of the pen holder 100, the foam 131 is exposed, the pen holder 100 is put into the mouth, and the foam 131 absorbs the saliva, the foam 131 can quickly absorb enough saliva; at this time, the saliva is all absorbed into the foam 131, did not enter the first cavity 101 to react with the test paper 120; then the front end of the pen holder 100 is inserted into the pen cap 200, and continue to be inserted when the front end of the pen holder 100 contacts the aluminum foil 220. The aluminum foil 220 is pierced, and the foam 131 absorbs the reaction solution in the base 210, the reaction solution is mixed with saliva; in the process of gradual insertion, the tight fit between the O-ring 134 and the inner wall of the cavity can prevent the mixture from overflowing when the foam 131 is squeezed;

At the same time, during the gradual insertion of the pen holder 100, the aluminum foil 220 is pierced, the inner wall of the cavity squeezes the foam 131, and the mixture (reaction solution+saliva) in the foam 131 enters the first cavity 101 through the channel and reacts with the test paper 120.

In particular, the foam 131 is squeezed inward by the inner wall of the cavity during the process of entering the cavity, and the mixed liquid is squeezed towards the channel.

In addition, due to the arrangement of the rubber stopper 111, after the pen holder 100 pierces the aluminum foil 220, due to the blocking effect of the rubber stopper 111, the saliva cannot be squeezed into the first cavity 101, giving the saliva and the reaction solution sufficient mixing/reaction time; finally, after the pen holder 100 is completely inserted into the pen cap 200, the front end of the foam support 130 is in contact with the inner bottom wall of the cavity, and the rubber stopper 111 is pushed to move backward, so that the mixture can smoothly enter the first cavity 101 and react with the test paper 120, and the gas in the first cavity 101 is discharged from the air hole 1121;

After reaching the detection time, the reaction results can be directly observed.

In addition, the test paper 120 can be of different types, and the reaction solution can also be of different types, which can be changed as the antigens to be detected are different. In this embodiment, the specific reaction principle between the test paper and the mixed solution is not made by detailed description.

The above description of the disclosed embodiments enables those skilled in the art to implement or use the present application. Various modifications to these embodiments will be readily apparent to those skilled in the art, and the generic principles defined herein may be implemented in other embodiments without departing from the spirit or scope of the present application. Thus, the present application is not intended to be limited to the embodiments shown herein, but comply with the widest scope consistent with the principles and novel features disclosed herein.

What is claimed is:

1. An antigenic detector, comprising: a pen holder (100) and a pen cap (200); wherein
the pen holder (100) comprises: a test paper tube (110), a test paper (120) assembled in the test paper tube (110), test paper supports (121), a foam support (130) installed on a front end of the test paper tube (110) and provided with a foam (131), and a protective cover (140);
the pen cap (200) includes: a base (210), and an aluminum foil (220) assembled in the base (210); an interior of the base (210) forms a cavity, configured to allow a front end of the pen holder (100) to be inserted to the cavity, and a reaction solution is sealed inside the cavity of the base (210) by the aluminum foil (220); the protective cover (140) is detachably installed on the front end of the test paper tube (110) and covers the foam (131);
the foam support (130) is provided with a channel that communicates with an interior of the test paper tube (110) such that a liquid absorbed by the foam (131) is mixed with the reaction liquid and is transferred to the test paper (120) inside the test paper tube (110) through the channel.

2. The antigenic detector according to claim 1, wherein the test paper tube (110) is in a shape of a hollow tube, the test paper supports (121) are arranged inside the test paper tube (110), and the test paper (120) is installed on test paper supports (121); and
an end of the test paper tube (110) is provided with a test tube cover (112), and an air hole (1121) communicating with the interior of the test paper tube (110) is defined on the test tube cover (112).

3. The antigenic detector according to claim 1, wherein the foam support (130) includes: an insertion end (132) inserted into the test paper tube (110) and a mounting end (133) extending out of the test paper tube (110); the foam (131) is mounted on the mounting end (133), and the foam (131) at least partially protrudes out of the test paper tube (110).

4. The antigenic detector according to claim 3, wherein an end of the mounting end (133) is a pointed structure.

5. The antigenic detector according to claim 1, wherein a rubber stopper (111) is arranged inside the test paper tube (110), and in anon-use state, the rubber stopper (111) holds the test paper (120) separated from foam support (130).

6. The antigenic detector according to claim 1, wherein the base (210) includes: an upper cover (211) and a lower cover (212); a boss (230) is formed on the lower cover (212), the boss (230) is provided with a sealing ring (231), and the lower cover (212) is inserted into the upper cover (211) through the boss (230).

7. The antigenic detector according to claim 6, wherein the foam support (130) is provided with an O-ring (134), and the O-ring (134) is in contact with an inner wall of the cavity of the base (210).

8. The antigenic detector according to claim 6, wherein the aluminum foil (220) is supported on the boss (230); when the upper cover (211) and the lower cover (212) are combined, the aluminum foil (220) is stably clamped, or the aluminum foil (220) is hot-pressed on the boss (230), or the aluminum foil (220) is ultrasonically fixed on the boss (230); or the aluminum foil (220) is glued to the boss (230).

9. The antigenic detector according to claim 1, wherein the test paper tube (110) is provided with an observation window or the test paper tube (110) is arranged to be transparent.

10. The antigenic detector according to claim 3, wherein the insertion end (132) of the foam support (130) is provided with an undercut (1321), and a buckle is arranged inside the test paper tube (110) corresponding to the undercut (1321) to prevent the foam support (130) from being pulled out of the test paper tube (110).
